(19) United States
(12) Patent Application Publication
(10) Pub. No.: US 2022/0354680 A1
(43) Pub. Date: Nov. 10, 2022

(54) SYSTEMS AND METHODS TO ENABLE PYLORIC CLOSURE

(71) Applicant: BOSTON SCIENTIFIC SCIMED, INC., MAPLE GROVE, MN (US)

(72) Inventors: Michael K. Peachock, Olmsted Falls, OH (US); Sean P. Fleury, Princeton, MA (US)

(73) Assignee: BOSTON SCIENTIFIC SCIMED, INC., MAPLE GROVE, MN (US)

(21) Appl. No.: 17/740,224

(22) Filed: May 9, 2022

Related U.S. Application Data
(60) Provisional application No. 63/186,333, filed on May 10, 2021.

(51) Int. Cl. A61F 5/00; A61B 17/29; A61B 17/11; A61B 5/06; A61B 90/30

(52) U.S. Cl. CPC A61F 5/0079; A61B 17/29; A61B 17/1114; A61B 5/061; A61B 90/30; A61F 5/0089; A61B 17/12031

(57) ABSTRACT

Devices, systems, and methods described herein relate to affecting an internal diameter of a body lumen, and, in many examples, of a pylorus. A silk-based bulking agent may be injected in a pyloric tissue so as to reduce an effective inner diameter of the pylorus. A multi-part occluding agent may be injected into a pylorus on the surface of the pyloric tissue to occlude the pylorus alone or in combination with the silk-based bulking agent.

### CROSS-REFERENCE TO RELATED APPLICATIONS

This application claims the benefit of priority of U.S. Provisional Application No. 63/186,333, filed May 10, 2021, the entire disclosure of which is hereby incorporated by reference herein for all purposes.

### FIELD

The present disclosure relates generally to the field of medical devices and methods for partially, temporarily, intermittently, and/or fully obstructing a body lumen. In particular, medical devices, systems, and methods are directed towards pyloric occlusion, such as during endoscopic metabolic procedures.

### BACKGROUND

Various medical approaches are used for treating bariatric or metabolic diseases, such as diet, medication, and surgical procedures. Surgical procedures such as bariatric surgery, e.g., to restrict a portion of a stomach and/or bypass portions of the intestine, may be the best option for some patients.

However, many bariatric procedures, such as restriction, sleeve gastrectomy, and biliopancreatic diversion with duodenal switch, may require irreversible rearrangement of the digestive system.

In Roux-en-Y procedures, an anastomosis is formed between a stomach pouch and small intestine, thereby allowing chyme to flow through the anastomosis and bypass the duodenum. Endoscopic metabolic procedures are an advantageous alternative to Roux-en-Y and may involve tools such as pyloric occlusion devices. However, pyloric occlusion devices such as expandable plugs may migrate, which may necessitate further procedures to correct positioning of the device.

Means for occluding body lumens with injectable media may cause irritation to tissue, may still be subject to migration, and/or may irreversibly damage tissue.

It is therefore desirable to provide a successful and minimally invasive alternative to existing approaches for treating bariatric or metabolic diseases.

With the above considerations in mind, a variety of advantageous medical outcomes may be realized by the devices and/or methods of the present disclosure.

### SUMMARY

In one aspect of the present disclosure, a method of occluding a pylorus may comprise advancing a catheter to the pylorus. The method may include creating at least one degradable pyloric implant by directly injecting a silk-based bulking agent into a pylorus, tissue adjacent to the pylorus, or both.

In various examples of the present disclosure, the silk-based bulking agent may be a tissue volumizing agent.

The silk-based bulking agent may adhere to surrounding tissue.

The direct injection may comprise injection without a preceding tissue priming step. The silk-based bulking agent may comprise a contrast agent.

The method may further comprise, after injecting the silk-based bulking agent, determining an extent of pyloric closure. The determination may be based on visualization via ultrasound or direct visualization. The method may further comprise, after determining the extent of pyloric closure, injecting an additional volume of the silk-based bulking agent to the pylorus, tissue adjacent to the pylorus, or both.

The silk-based bulking agent may be injected in two or more locations about a circumference of a pylorus. The injections in the two or more locations may create longitudinally extending straightened surfaces corresponding to the two or more locations. The longitudinally extending straightened surfaces may be configured to contact one another. The longitudinally extending straightened surfaces may be configured to contact one another so as to limit flow therebetween.

The injection of the silk-based bulking agent may result in a reduction of an internal diameter of a pylorus. The reduction may be sufficient to limit flow of chyme through the pylorus. The injection of the silk-based bulking agent may result in a complete sealing of the pylorus.

The silk-based bulking agent may be a silk-based fibrin protein in aqueous solution.

The silk-based bulking agent may comprise a hyaluronic acid carrier.

The silk-based bulking agent may comprise an in-situ hydrogel.

The injecting of the silk-based bulking agent may result in a permanent volumization of the pylorus, tissue adjacent to the pylorus, or both.

The silk-based bulking agent may comprise at least one growth factor.

The degradable pyloric implant may be configured to degrade at approximately a same rate as a growth rate of an apposed tissue.

The method may further comprise injecting, from the catheter, a first adhesive agent and a second adhesive agent onto at least one side of the pyloric sphincter. The first adhesive agent and the second adhesive agent may be configured to react to polymerize and form an intraluminal occlusion device within the pylorus. The intraluminal occlusion device may be configured to appose the tissue injected with the silk-based bulking agent. The intraluminal occlusion device may be anchored to the at least one side of the pyloric sphincter.

The method may further comprise injecting the silk-based bulking agent with a first injection device and injecting the first adhesive agent and the second adhesive agent with a second injection device, the second injection device optionally being a single- or dual-channel injection device.

The method may further comprise curing to affect the polymerization of the silk-based bulking agent, of the intraluminal occlusion device, or both. The curing may comprise UV activation, heat activation, other activation method, or any combination thereof.

The first adhesive agent may be fibrinogen.

The second adhesive agent may be thrombin. The method may further comprise, after the injection of the first and second adhesive agents, determining an extent of pyloric closure using ultrasound.

The injecting of the first agent and the second agent may be performed as two sequential steps. The injecting of the first agent and the second agent may be performed in synchronous coordination.

The intraluminal occlusion device may reduce an inner diameter of the pylorus sufficiently to limit flow of chyme through the pylorus. The intraluminal occlusion device may completely seal the pylorus.

The first adhesive agent and the second adhesive agent may be further injected about a circumference of the pyloric sphincter.

The occlusion device may comprise an hourglass shape.

In another aspect of the present disclosure, a method of occluding a pylorus may comprise advancing a catheter to the pylorus and, from the catheter, injecting a fluid silk-based agent into a mucosa, submucosa, muscularis, or a combination thereof of the pylorus. The fluid silk-based agent may be configured to create a degradable tissue scaffold within the mucosa, submucosa, muscularis, or combination thereof.

According to one or more examples of the present disclosure, the fluid silk-based agent may be a tissue volumizing agent.

The fluid silk-based agent may be configured to adhere to surrounding tissue.

The injection may comprise injection without a preceding tissue priming step.

The fluid silk-based agent may comprise a contrast agent.

The method may comprise, after injecting the fluid silk-based agent, determining an extent of pyloric closure. The determination may be based on visualization via ultrasound or direct visualization. The method may further comprise, after determining the extent of pyloric closure, injecting an additional volume of the fluid silk-based agent to the pylorus, tissue adjacent to the pylorus, or both.

The fluid silk-based agent may be injected in two or more locations about a circumference of a pylorus. The injections in the two or more locations may create longitudinally extending straightened surfaces corresponding to the two or more locations, the longitudinally extending straightened surfaces configured to contact one another. The longitudinally extending straightened surfaces may be configured to contact one another so as to limit flow therebetween.

The injection of the fluid silk-based agent may result in a reduction of an internal diameter of a pylorus. The reduction may be sufficient to limit flow of chyme through the pylorus. The injection of the fluid silk-based agent may result in a complete sealing of the pylorus.

The fluid silk-based agent may be a silk-based fibrin protein in aqueous solution.

The fluid silk-based agent may comprise a hyaluronic acid carrier.

The fluid silk-based agent may comprise an in-situ hydrogel.

The injecting of the fluid silk-based agent may result in a permanent volumization of the pylorus, tissue adjacent to the pylorus, or both.

The fluid silk-based agent may comprise at least one growth factor.

The degradable tissue scaffold may be configured to degrade at approximately a same rate as a growth rate of an apposed tissue.

The method may include injecting, from the catheter, a first adhesive agent and a second adhesive agent onto at least one side of the pyloric sphincter, the first agent and the second agent configured to react to polymerize and form an intraluminal occlusion device within the pylorus and apposing the tissue injected with the silk-based bulking agent, the occlusion device anchored to the at least one side of the pyloric sphincter. The method may further include injecting the silk-based bulking agent with a first injection device and injecting the first adhesive agent and the second adhesive agent with a second injection device. The second injection device may be a single-channel injection device. The second injection device may alternatively be a dual-channel injection device.

The method may comprise curing to affect a polymerization of the fluid silk-based agent, of the intraluminal occlusion device, or both. The curing may comprise UV activation, heat activation, other activation method, or any combination thereof.

The first adhesive agent may be fibrinogen, the second adhesive agent may be thrombin, or both.

The method may include, after the injection of the first and second adhesive agents, determining an extent of pyloric closure using ultrasound.

The injecting of the first agent and the second agent may be performed as two sequential steps. The injecting of the first agent and the second agent may be performed in synchronous coordination.

The intraluminal occlusion device may reduce an inner diameter of the pylorus sufficient to limit flow of chyme through the pylorus. The intraluminal occlusion device may completely seal the pylorus. The first adhesive agent and the second adhesive agent may be further injected about a circumference of the pyloric sphincter. The occlusion device may comprise an hourglass shape.

In yet another aspect of the present disclosure, a tissue scaffold for occluding a pylorus may be formed of a silk-based tissue volumizing agent injected directly into a mucosa of a pylorus, submucosa of the pylorus, muscularis of the pylorus, or a combination thereof.

According to various examples of the present disclosure, the silk-based tissue volumizing agent may be configured to adhere to the mucosa of a pylorus, submucosa of the pylorus, muscularis of the pylorus, or combination thereof.

The injection may comprise injection without a preceding tissue priming step.

The silk-based tissue volumizing agent may comprise a contrast agent.

The silk-based tissue volumizing agent may be imageable via ultrasound.

The tissue scaffold may be formed by injecting the silk-based tissue volumizing agent in two or more locations about a circumference of a pylorus.

The tissue scaffold may be configured to distend the pylorus, muscularis of the pylorus, or combination thereof so as to create at least two articulating surfaces thereof. The at least two articulating surfaces may be longitudinally extending straightened surfaces. The at least two articulating surfaces may be configured to contact one another so as to limit flow therebetween.

The tissue scaffold may be configured to reduce an internal diameter of a pylorus. The reduction may be sufficient to limit flow of chyme through the pylorus. The tissue scaffold may be configured to bulk the pylorus so as to completely seal the pylorus.

The silk-based tissue volumizing agent may be a silk-based fibrin protein in aqueous solution.

The silk-based tissue volumizing agent may comprise a hyaluronic acid carrier.

The silk-based tissue volumizing agent may comprise an in-situ hydrogel.

The silk-based tissue volumizing agent may be configured to generate a permanent volumization of the pylorus, tissue adjacent to the pylorus, or both.

The silk-based tissue volumizing agent may comprise at least one growth factor.

The tissue scaffold may be configured to degrade at approximately a same rate as a growth rate of an apposed tissue.

In at least one aspect of the disclosure, a system for occluding a pylorus may comprise a catheter, an injection device disposed within a distal end of the catheter a fluid silk-based volumizing agent. The injection device may be configured to dispose the fluid silk-based volumizing agent into a mucosa of the pylorus, submucosa of the pylorus, muscularis of the pylorus, or a combination thereof. The fluid silk-based volumizing agent may be configured to polymerize to form an in-situ scaffold. The scaffold may be configured to degrade at a same rate as a growth rate of the mucosa of the pylorus, submucosa of the pylorus, muscularis of the pylorus, or combination thereof.

In this and in other aspects of the disclosure, the silk-based tissue volumizing agent may be configured to adhere to the mucosa of a pylorus, submucosa of the pylorus, muscularis of the pylorus, or combination thereof.

The silk-based volumizing agent may be configured to be injected without a preceding tissue priming step.

The silk-based tissue volumizing agent may comprise a contrast agent.

The silk-based tissue volumizing agent may be imageable via ultrasound.

The tissue scaffold may be formed by injecting the silk-based tissue volumizing agent in two or more locations about a circumference of a pylorus.

The tissue scaffold may be configured to distend the pylorus, muscularis of the pylorus, or combination thereof so as to create at least two articulating surfaces thereof. The at least two articulating surfaces may be longitudinally extending straightened surfaces. The at least two articulating surfaces may be configured to contact one another so as to limit flow therebetween.

The tissue scaffold maybe configured to reduce an internal diameter of a pylorus. The reduction may be sufficient to limit flow of chyme through the pylorus. The tissue scaffold may be configured to bulk the pylorus so as to completely seal the pylorus.

The silk-based tissue volumizing agent may be a silk-based fibrin protein in aqueous solution.

The silk-based tissue volumizing agent may comprise a hyaluronic acid carrier.

The silk-based tissue volumizing agent may comprise an in-situ hydrogel.

The silk-based tissue volumizing agent may be configured to generate a permanent volumization of the pylorus, tissue adjacent to the pylorus, or both.

The silk-based tissue volumizing agent may comprise at least one growth factor.

The tissue scaffold may be configured to be entirely replaced by tissue.

In an additional aspect, a pyloric occlusion device may comprise a tissue scaffold formed in-situ, the tissue scaffold formed of a silk-based bulking agent injected directly into a mucosa of a pylorus, submucosa of the pylorus, muscularis of the pylorus, or a combination thereof.

In a further aspect of the present disclosure, an injectable pyloric occlusion medium may comprise a silk-based tissue volumizing agent configured to self-polymerize into a pliable bolus degradable in a pyloric tissue at approximately a same rate as a growth rate of the pyloric tissue.

In yet another aspect of the disclosure, a method of occluding a pylorus may comprise advancing a catheter to a pyloric sphincter and injecting, from the catheter, a first agent and a second agent onto at least one side of the pyloric sphincter. The first agent and the second agent may be configured to react to polymerize and form an occlusion device within the pylorus. The occlusion device may be anchored to the at least one side of the pyloric sphincter.

In this and in various other aspects, the first agent may be fibrinogen, the second agent may be thrombin, or both.

The method may further include, after injecting the first agent and the second agent, determining an extent of pyloric closure using ultrasound.

The injecting of the first agent and the second agent may be performed as two sequential steps.

The first agent and the second agent may be injected using a dual-channel injection device.

The injecting of the first agent and the second agent may be performed in synchronous coordination.

The occlusion device may reduce an inner diameter of the pylorus sufficient to limit flow of chyme through the pylorus. The occlusion device may completely seal the pylorus.

The first agent and the second agent may be injected about a circumference of the pyloric sphincter.

The occlusion device may comprise an hourglass shape.

The method may further include curing to affect the polymerization. The curing may comprise UV activation, heat activation, or other activation method.

In an additional aspect, a pyloric occlusion device may be formed within a pylorus by a first and second agent polymerizing about a circumference of at least one side of a pyloric sphincter. The pyloric occlusion device may extend radially towards a longitudinal axis extending through a center of the pyloric sphincter so as to reduce an inner diameter of a lumen defined by the pyloric sphincter.

In a further aspect of the present disclosure, a system for occluding a pylorus may comprise a silk-based bulking agent. The system may include a first injection device comprising a first device channel extending therethrough. The first device channel may contain the silk-based bulking agent. The first injection device may include a first injection device controller configured to iteratively expel the silk-based bulking agent from the first device channel. The system may include a first sealant agent and a second sealant agent. The second sealant agent may be configured to polymerize when placed in contact with the first sealant agent. The system may include a second injection device. The second injection device may include at least one second device channel extending therethrough, the second device channel comprising the first sealant agent, the second sealant agent, or both. The second injection device may include a second injection device controller configured to iteratively expel the first sealant agent, the second sealant agent, or both from the at least one second device channel. The system may include at least one catheter comprising at least one working channel configured to accommodate one or both of the first or second injection devices.

According to at least one aspect of the present disclosure, a system for occluding a pylorus may comprise a silk-based bulking agent injected into a pyloric tissue and a fibrin glue deposited onto a surface of the pyloric tissue.

According to the above and other aspects of the present disclosure, the fibrin glue may be configured to form an in-situ implant. The in-situ implant may completely occlude the pylorus.

The silk-based tissue volumizing agent may comprise a hyaluronic acid carrier.

The silk-based tissue bulking agent may comprise an in-situ hydrogel.

The silk-based tissue bulking agent may be configured to generate a permanent volumization of the pylorus, tissue adjacent to the pylorus, or both.

The silk-based tissue bulking agent may comprise at least one growth factor.

The silk-based bulking agent may be configured to degrade at approximately a same rate as a growth rate of an apposed tissue.

The fibrin glue may be removable.

The silk-based bulking agent may partially occlude the pylorus.

The silk-based bulking agent may be injected prior to depositing of the fibrin glue. The silk-based bulking agent may be injected after depositing of the fibrin glue.

One or both of the silk-based bulking agent and the fibrin glue may be imageable. The one or both of the silk-based bulking agent and the fibrin glue may be imageable via ultrasound.

In a further aspect of the present disclosure, a system for occluding a pylorus may comprise a silk-based volumizing agent and a first injection device configured to inject fluid into a tissue. The first injection device may comprise a first device channel extending therethrough, the first device channel containing the silk-based bulking agent. The system may include a first bulking agent component. The system may include a second bulking agent component configured to polymerize when placed in contact with the first bulking agent component. The system may include a second injection device configured to deposit fluid onto a tissue surface. The second injection device may comprise at least one second device channel extending therethrough, the at least one second device channel comprising the first bulking agent component, the second bulking agent component, or both.

In an additional aspect of the present disclosure, a system for occluding a pylorus may include a volumizing agent and a first bulking agent component. The system may include a second bulking agent. The second bulking agent component may be configured to polymerize when placed in contact with the first bulking agent component. The system may include at least one injection device. The at least one injection device may comprise at least one channel extending therethrough. The at least one injection device may be configured to inject the volumizing agent into a tissue. The at least one injection device may be configured to deposit the first bulking agent component and the second bulking agent component onto the tissue surface.

In the above and other aspects of the disclosure, the tissue may be a pylorus.

The first bulking agent component may be thrombin and the second bulking agent component may be fibrinogen.

The volumizing agent may be a silk-based volumizing agent.

The volumizing agent may be configured to degrade at approximately a same rate as a growth rate of the tissue.

The volumizing agent may be configured to, when injected into the tissue, augment the tissue.

The tissue may define a lumen. The volumizing agent may be configured to, when injected into the tissue, effect a partial occlusion of the lumen.

The first and second bulking agent components may be configured to polymerize to form an in-situ implant. The in-situ implant may be configured to fully occlude the lumen.

The in-situ implant may be removable.

In another aspect of the present disclosure, a pyloric occlusion implant may include a first bulking agent component and a second bulking agent component. The second bulking agent may be configured to polymerize when placed in contact in-situ with the first bulking agent component on a surface of a pyloric tissue. The pyloric occlusion implant may be configured to adhere to the pyloric tissue upon the polymerization of the first bulking agent component with the second bulking agent component.

In this and various other aspects, the pyloric occlusion implant may comprise an hourglass shape.

The pyloric occlusion device may be configured to fully occlude a pylorus.

The pyloric occlusion device may be configured to be removable via resection.

The pyloric occlusion device may be formed during a single procedure. The pyloric occlusion device may be formed during multiple procedures.

The first bulking agent component may be thrombin and the second bulking agent component may be fibrinogen.

The pyloric occlusion device may be configured to accommodate a natural shape of the tissue.

The pyloric occlusion implant may be formed about a circumference of the pyloric sphincter.

In an additional aspect, an injectable pyloric occlusion medium may comprise a silk-based tissue volumizing agent configured to create a tissue scaffold when injected into a pyloric tissue. The tissue scaffold may be configured to degrade at approximately a same rate as a growth rate of the pyloric tissue.

According to at least this aspect, the silk-based tissue volumizing agent may be a silk-based fibrin protein in aqueous solution.

The silk-based tissue volumizing agent may comprise a hyaluronic acid carrier.

The silk-based tissue volumizing agent may comprise an in-situ hydrogel.

The tissue scaffold may be configured to result in a permanent volumization of the pyloric tissue.

In a further aspect, a pyloric occlusion system may comprise a silk-based tissue volumizing agent injected into a pyloric tissue. The silk-based tissue volumizing agent may be configured to degrade at approximately a same rate as a growth rate of the pyloric tissue.

According to the above and other aspects, the silk-based tissue volumizing agent may be configured to partially occlude a pylorus.

The pyloric occlusion system may further comprise a first bulking agent component and a second bulking agent component configured to polymerize when placed in contact in-situ with the first bulking agent component on a surface of the pyloric tissue. The first agent may be fibrinogen, the second agent may be thrombin, or both. The first bulking agent component and the second bulking agent may be configured to polymerize to form an in-situ implant. The in-situ implant may be configured to completely occlude the pylorus. The in-situ implant may comprise an hourglass shape. The in-situ implant may be configured to anchor to at least one side of the pyloric sphincter.

The silk-based tissue volumizing agent may be imageable via ultrasound.

The silk-based tissue volumizing agent may be a silk-based fibrin protein in aqueous solution.

The silk-based tissue volumizing agent may comprise a hyaluronic acid carrier.

The silk-based tissue volumizing agent may comprise an in-situ hydrogel.

In an additional aspect, a method of occluding a pylorus may comprise advancing a catheter to the pylorus. The method may include creating at least one degradable pyloric implant by directly injecting a bulking agent into a pyloric tissue, wherein the bulking agent is selected to degrade at approximately a same rate as a growth rate as tissue of the pyloric tissue.

In another aspect of the present disclosure, an injectable pyloric occlusion implant may include a first bulking agent component and a second bulking agent component. The second bulking agent may be configured to polymerize when placed in contact in-situ with the first bulking agent component on a surface of a pyloric tissue.

In one aspect of the present disclosure, an injectable pyloric occlusion medium may include a silk-based bulking agent. The injectable pyloric occlusion medium may be selected to degrade at approximately a same rate as a growth rate as tissue of a pyloric tissue.

In one aspect of the present disclosure, an injectable pyloric occlusion medium may include a silk-based bulking agent. The silk-based bulking agent may be selected to degrade at approximately a same rate as a growth rate as tissue of a pyloric tissue.

In one or more additional aspects of the present disclosure, an injectable pyloric occlusion medium may include a bulking agent. The bulking agent may be selected to degrade at approximately a same rate as a growth rate as tissue of a pyloric tissue.

In a further aspect, a system for occluding a pylorus may comprise an injection device and an injectable pyloric occlusion medium. The injectable pyloric occlusion medium may comprise a silk-based bulking agent.

In the above and other aspects, the silk-based bulking agent may comprise a degradation rate approximately the same as a growth rate of an apposed tissue.

The system may further comprise a first adhesive agent.

The system may further comprise a second adhesive agent. The first agent and the second agent may be configured to react with each other to polymerize and form an intraluminal occlusion device within the pylorus. The occlusion device may be configured to be anchored to at least one side of the pyloric sphincter.

The system may further comprise an additional injection device.

Combinations of one or more discussed features are presently contemplated, as will be clear to one of skill in the art.

### BRIEF DESCRIPTION OF THE SEVERAL VIEWS OF THE DRAWINGS

Non-limiting embodiments of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1A illustrates an expanded cross-section view of a pylorus depicting a first positioning of an injection device for injection of a bulking agent according to various embodiments described herein.

FIG. 1B illustrates an additional expanded cross-section view of the pylorus depicting a first injection of a bulking agent according to various embodiments described herein.

FIG. 1C illustrates an additional expanded cross-section view of the pylorus depicting an additional injection of a bulking agent according to various embodiments described herein.

FIG. 2A illustrates an expanded cross-section view of a pylorus depicting a second positioning of an injection device for injection of a bulking agent according to various embodiments described herein.

FIG. 2B illustrates an additional expanded cross-section view of the pylorus depicting a first injection of a bulking agent according to various embodiments described herein.

FIG. 2C illustrates an additional expanded cross-section view of the pylorus depicting an additional injection of a bulking agent according to various embodiments described herein.

FIG. 3A illustrates multiple discontinuously arranged injections of a bulking agent about a circumference of a body lumen according to various embodiments described herein.

FIG. 3B illustrates multiple continuously arranged injections of a bulking agent about a circumference of a body lumen according to various embodiments described herein.

FIG. 4 illustrates an expanded cross-section view of a pylorus depicting use of an injection device for a first deposition of a bulking agent along a pyloric sphincter according to various embodiments described herein.

FIG. 5 illustrates an expanded cross-section view of a pylorus depicting one or more injections of a bulking agent into a pyloric sphincter and deposition of a bulking agent on a pyloric sphincter according to various embodiments described herein.

FIG. 6 illustrates an expanded cross-section view of a pylorus depicting use of an injection device for a second deposition of a bulking agent along a pyloric sphincter according to various embodiments described herein.

### DETAILED DESCRIPTION

The present disclosure is related to devices, systems, and methods for occluding, limiting, or otherwise facilitating a regulated fluid flow such as between body lumens. Various embodiments include using one or more injectable bulking agents to affect a profile of a tissue surface defining a body lumen.

For example, natural orifice transluminal endoscopic surgery (NOTES) procedures may be advantageous over other types of bariatric procedures, such as gastric procedures, by enabling a redirection of flow (e.g., chyme or other gastric flow) via an anastomosis (e.g., created between the stomach and a jejunal loop of small bowel in the jejunum). However, redirection of flow may necessitate an occlusion of a natural flow path in favor of the surgically introduced alternative path.

Devices, systems, and methods described herein may assist in fluid flow path occlusion, and in particular occlusion of gastric flow into the duodenum, which may limit digestion of food, liquid, and other nutrients until further down the GI tract. Many embodiments may relate to endoscopic ultrasound procedures. In some embodiments, a pyloric closure or occlusion agent may be reversible, e.g., a medical professional, physician, and/or automated system may be able to deliver and/or remove the agent endoscopically. Although the devices, systems, and methods are described herein with respect to a gastrointestinal system, it will be understood that illustrative embodiments and/or elements and/or features of devices, systems, and methods in accordance with various principles of the present disclosure may be advantageous for use in any other procedures and/or anatomy, to prevent movement of material such as through a body lumen.

In embodiments of the present disclosure, at least one injectable occlusion agent may be used to control or limit flow through a body lumen, and in many examples, a pylorus.

Several existing injectable agents may be used to augment a tissue. For example, solid implants have been formed by injecting various polymers into a tissue wall. However, many traditional methods, devices, and systems include such limitations as: (a) requiring a priming saline injection step prior to the introduction of an implant; (b) having a higher stiffness than surrounding tissue, which may cause the implant to irritate surrounding tissue, alter the ability of the surrounding tissue to operate with regular muscular strength and/or elastic response to stimuli, or both; (c) leading to inflammation of surrounding tissue; (d) tending to migrate from an initial placement site, for example, in response to a natural motion of a patient's body; and/or (e) sufficiently permanently augmenting a tissue so as to prevent reversing of a procedure. Each of these deficiencies may, alone or in combination, contribute to risk of discomfort and/or undesirable tissue damage for a patient.

Devices, systems, and methods presently disclosed may remedy one or more of the above deficiencies. In various embodiments discussed herein, a bulking agent, tissue volumizing agent, or the like may be injected into or otherwise introduced underneath a tissue layer. For example, a tissue bulking agent may be introduced in and/or under a layer of mucosa, submucosa, or muscularis of a tissue, and in many examples, of a pyloric tissue. For the sake of simplicity, and without intent to limit, references herein to pyloric tissue are intended to encompass references to the tissue of the pyloric sphincter, the pyloric antrum, and/or the pyloric sphincter and surrounding/adjacent/nearby tissue as appropriate. A tissue bulking agent may comprise an in-situ hydrogel or other material with a significant dwell time, and in some examples, a silk-based fibroin protein. The tissue bulking agent may comprise an aqueous solution, and in some cases a biocompatible carrier such as hyaluronic acid.

Without wishing to be bound by any particular theory, it is believed that certain tissue bulking agents, such as silk-based tissue bulking agents, may support an increase in tissue volume and/or remodeling of surrounding tissue, thereby enabling a longer-lasting and lower risk method of augmenting a tissue than alternative methods, for example, bulking agents such as autologous fat, glutaraldehyde, cross-linked bovine collagen, calcium hydroxylapatite, prolytic carbon-coated beads/microspheres, polydimethylsiloxane, ethylene vinyl alcohol copolymer, dextranomer hyaluronic acid, calcium alginate, polytetrafluoroethylene and polymethylmethacrylate.

For example, in accordance with various principles of the present disclosure, a bulking agent, such as a silk-based tissue bulking agent, may be selected and/or configured to form an in-situ implant when injected in or under a tissue, wherein the implant thereby formed is configured to degrade at a similar or an approximately equal rate as a growth rate of the tissue in which the bulking agent has been implanted. Without wishing to be bound by any particular theory, it is believed that the implant may form a degradable scaffold for fibrinogens, and/or that, as the implant degrades over time, it may facilitate a corresponding in-growth of surrounding tissue such that the space once occupied by the implant is subsequently filled by a natural tissue of the patient. To this end, a “volumizing agent” as referred to herein may distinguish above traditional injected/implanted agents by facilitating a volumizing of a patient's own tissue over time, rather than, for example, merely augmenting a tissue shape by creating a permanent synthetic platform upon which a tissue may rest (i.e., a submucosal injection of a substantially permanent glue, polymer, or other material).

Without wishing to be bound by any particular theory, it is believed that injections of bulking or volumizing agents as discussed herein may result in lower risks of implant migration and/or tissue irritation than traditional systems and methods. For example, particular degradation rate(s) of silk-based bulking agents may enable corresponding tissue ingrowth to result in the integration of an amount of non-degraded scaffold (i.e., remnant of partially degraded silk-based bulking agent) into surrounding tissue, which may reduce a risk of migration of the implant over traditional methods. In the same or in another example, the integration of a patient's own tissue into the scaffold structure and eventual replacement of the volume of volumizing agent with the patient's own tissue may result in lower risk of irritation or inflammation of tissue than traditional systems and methods, for example, which augment a tissue shape with a foreign substrate permanently placed in tissue. Based on lower risks of irritation or inflammation as opposed to traditional methods, various embodiments as described herein may have a lower risk of rejection as opposed to traditional methods (i.e., via triggered immune response).

In various embodiments in accordance with aspects of the present disclosure, an injectable tissue bulking or volumizing agent, such as a silk-based bulking agent, may be used to augment a tissue so as to reduce an internal diameter of a body lumen defined by the tissue. For example, a silk-based bulking agent may be used to create one or more implants about a circumference of a pyloric tissue, for example, a pyloric sphincter, such as to reduce an internal diameter of the pylorus. In some embodiments, a silk-based bulking agent may be used to close and/or substantially seal a body lumen, for example, by creating tissue-volumizing implants about a circumference of a pyloric tissue so as to reduce an inner diameter of the pylorus to a point of closure.

A tissue bulking or volumizing agent may be iteratively injected in a tissue so as to ensure appropriate reduction of an inner diameter of a body lumen without waste of material, excess closure of the lumen, or the like. For example, a silk-based bulking agent and/or the bulking effect thereof in a tissue may be visualizable via an imaging system, for example, ultrasound. In various examples, a first amount of silk-based bulking agent may be injected in a tissue defining a body lumen, at which point a degree of occlusion of the body lumen may be determined, for example, via direct visualization (e.g., via a camera) or via imaging (e.g., via ultrasound). If a greater degree of occlusion is desired, a second amount of silk-based bulking agent may be injected. In this example, the first and second injections may be performed in the same or in different procedures. For example, a patient's needs may develop over time such that a greater degree of occlusion is desired over an initial partial occlusion, in which case, a secondary procedure may be used to inject an additional amount of volumizing agent. In some embodiments, a silk-based bulking agent may comprise a contrast agent to aid in visualization.

In accordance with various principles of the present disclosure, additionally or alternatively, an occlusive agent (e.g., a sealant, adhesive agent, or the like, which may be a multi-agent sealant) may be injected and/or deposited about a circumference of the pylorus (e.g., on the surface of the pylorus, rather than being injected into the pylorus) to secure and/or contribute to a closure of the pylorus. Such occlusive agent may be sufficient to occlude the pylorus without the use of a tissue bulking or volumizing agent, or may be used in conjunction with a tissue bulking or volumizing agent. If a tissue bulking or volumizing agent, such as those described herein, is used, then the tissue bulking or volumizing agent (e.g., silk-based bulking agent) may be injected into the tissue of the pylorus to create an initial partial or complete closure of the pylorus, and an occlusive agent may subsequently be injected and/or deposited over the tissue area or region in which the tissue bulking or volumizing agent had been injected. Reference is made herein to occlusive agents for the sake of convenience and without intent to limit to refer to surface-applied agents for occluding flow through a body lumen, in contrast with the previously described agents injected into the body tissue.

For example, an occlusive agent may be injected on or over tissue (e.g., on the surface of the tissue, in contrast with into the tissue) to form an in-situ plug entirely occluding a pylorus. It will be understood that deposition of occlusive agents (e.g., sealants, adhesive agents, or the like) on a surface of a tissue surface as described herein may enable in situ formation of occlusive implants, plugs, or the like, which may be formed on and/or adhere directly to the tissue surface. While various types of occlusive agents are presently contemplated, various sealants and/or adhesives, such as multi-agent sealants, may enable specific customization of in situ implant properties. For example, various ratios of reactive components may be used to adjust a pliability, hydrophobicity, adherence, or the like of a resultant implant. In various embodiments in accordance with various principles of the present disclosure, an occlusive agent may include a fibrin glue, with a first part comprising fibrinogen and a second part comprising thrombin.

In various examples, an occlusive agent may be disposed in one or more locations on a circumference of a pyloric tissue, such as a pyloric sphincter. In some examples, the multi-part occlusive agent may be disposed on a side of a pyloric sphincter and may further span an opening thereof. In various examples, a multi-part occlusive agent may be disposed about a circumference of a tissue adjacent to the pyloric tissue. It will be understood that adhesion of the multi-part occlusive agent to multiple surface areas of the pyloric sphincter may resist tendency of the formed in-situ implant to migrate.

Occlusion of a tissue lumen, for example, a pylorus, with an implant formed by injecting an occlusive agent, such as a multi-part occlusive agent, on a tissue surface may enable reversibility of the procedure. For example, an implant comprising an occlusive agent on a tissue surface may be resected more easily than an implant injected in or under a tissue surface. Furthermore, resection of and/or other removal of an implant formed from an occlusive agent provided (e.g., adhered) on a tissue surface may avoid damaging the tissue, or reduce damage as compared to an attempt to resect an implant created underneath a tissue surface.

Coordinated use of an injected bulking agent with an occlusive agent provided on the tissue surface may present one or more advantages over using singular traditional methods. For example, the injected bulking agent may be used to partially occlude a patient's pylorus insofar as a medical professional is confident that permanent narrowing of the pylorus may be warranted. However, in the event that the medical professional may wish to further occlude the pylorus immediately but reserve the option to later reopen the lumen, an additional occlusive agent may be provided on the tissue surface to create a removable occlusive implant. Upon reexamination, the medical professional may then determine that the removable occlusive implant should be removed partially or entirely (e.g., by resection) or that additional silk-based bulking agent should be injected in order to increase a permanence of the occlusion.

Without wishing to be bound by any particular theory, it is further believed that combinations of use of injectable agents both under a tissue surface (e.g., a silk-based bulking agent injected into a pyloric sphincter) and on a tissue surface (e.g., a fibrin glue applied on a surface of the pyloric sphincter) may additively contribute to occlude a lumen associated with the tissue surface.

For example, a fibrin glue or other multi-part occlusive agent may be used to create a cap over a tissue injected with a silk-based bulking agent. The fibrin glue may polymerize (more quickly than the silk-based bulking agent) to stabilize the tissue so as to allow the silk-based bulking agent to set in a desired position within the tissue, with decreased risk of migration or deformation of the silk-based bulking agent prior to complete polymerization of the same. It will be further understood that, post-polymerization of the silk-based bulking agent, a cap created by a fibrin glue or other sealing agent may reduce a risk of post-operative migration of the implant created with silk-based bulking agent, for example, as a result of a patient's body motion such as involuntary motion of a pylorus.

In the same or another example, the injected silk-based bulking agent may initially stabilize and/or position a tissue such that the fibrin glue may be applied to hold a position of the tissue. It will be understood that the bulking of the tissue via the injected silk-based bulking agent may further reduce a post-operative risk of the migration of the fibrin glue implant, for example, as a result of a patient's body motion (e.g., involuntary motions of the pylorus).

Accordingly, combinations of injectable occlusive agents discussed herein may address various weaknesses of traditional methods of occluding a lumen such as a pylorus, presenting collaborative benefits of each of various agents applied within and on a surface of a tissue to, among other benefits, enable a more precise, stable, and/or durable placement of one or more injectable agents for occluding a lumen, for example, a pylorus.

It is further currently contemplated that, in some examples, a permanent occlusion of a lumen may not be desired. In such examples, among others, an injected occlusive agent, such as fibrin glue, may be used without a corresponding injection of a silk-based bulking agent. According to various principles of the present disclosure, a fibrin glue may be used to create an in-situ partial or complete plug of a body lumen, for example, a pylorus (e.g., which partially or completely occludes the body lumen). It will be understood that injection of the occlusive agent so as to accommodate and/or conform to the contours of a surface of an adjacent tissue and, in various examples, filling of an intervening space defined by a circumference of the surface, may contribute to an increased area of adhesion of the plug to the surface, thereby reducing a risk of migration. For example, a fibrin glue may be applied about a pyloric sphincter in a generally hourglass shape. A plug formed in-situ via a sealant as presently discussed may present various embodiments over alternative implanted methods of occluding a lumen. For example, a fibrin glue plug may be less costly to produce than a stent designed to occlude a pylorus and/or may have a lower risk of migration.

Accordingly, various embodiments presented herein may be understood to present various benefits over traditional methods of occluding a body lumen, and, in particular, a pylorus. Attention will now be given to the drawings. The detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and “distal” refers to the direction or location furthest from the user, such as when using the device (e.g., introducing the device into a patient, or during implantation, positioning, or delivery). “Longitudinal” means extending along the longer or larger dimension of an element. “Central” means at least generally bisecting a center point, and a “central axis” means, with respect to an opening, a line that at least generally bisects a center point of the opening, extending longitudinally along the length of the opening when the opening comprises, for example, a tubular element, a channel, a cavity, or a bore.

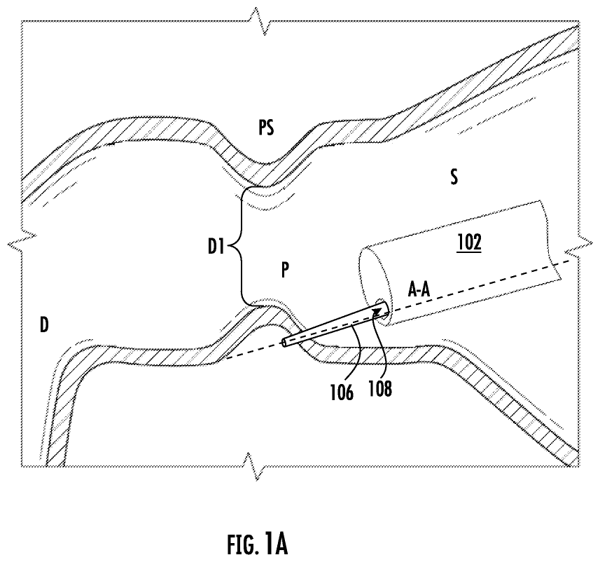

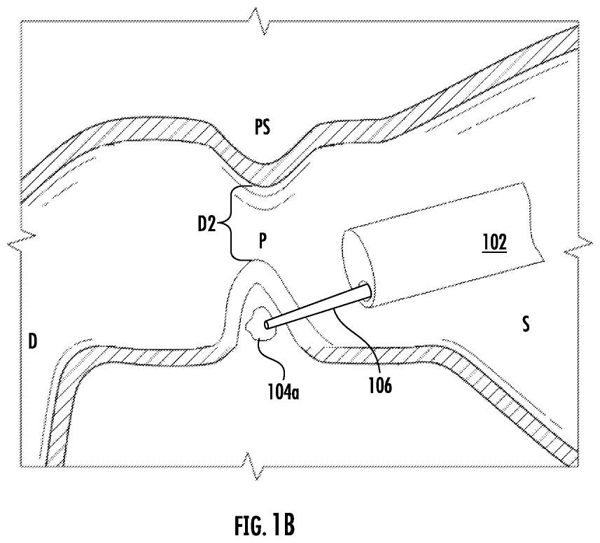

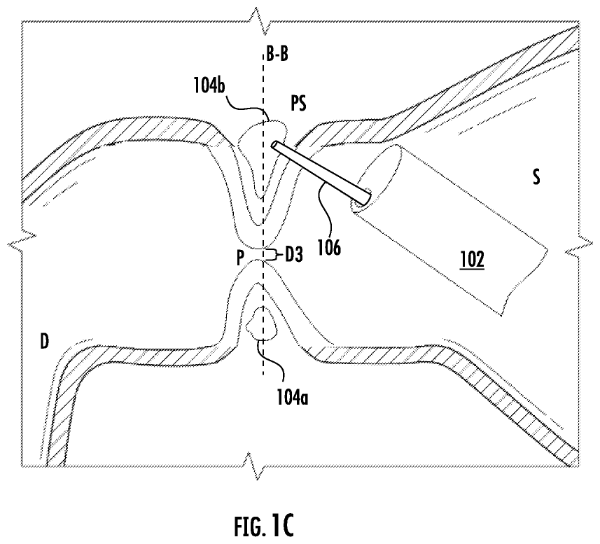

Referring now the drawings for illustrations of examples embodying various of the above-described aspects and principles, FIGS. 1A-1C illustrate a human digestive system as an example of an environment for tightening, restricting, or otherwise occluding a body lumen using an injection device 106. In particular, the area labeled “S” corresponds to a stomach, the area labeled “P” corresponds to a pylorus, the anatomical feature “PS” corresponds to a pyloric sphincter, and the area labeled “D” corresponds to a duodenum. In an open, or non-occluded, system, chyme comprising digestive fluids and food particles may pass from the stomach through the pylorus and into the duodenum. However, in various procedures, such as a Roux-en-Y, there may be a desire to redirect or limit digestive flow through the pylorus.

Accordingly, a catheter 102 comprising at least one working channel 108 may be advanced to a pylorus P. Catheter 102 may be coupled to a proximal handle (not shown), which may be used to position and/or operate catheter 102 and/or at least one injection device 106 extending through a working channel 108 extending through the catheter 102 along a longitudinal axis “A-A” thereof. For example, injection device 106 may comprise an injection needle. Injection device 106 may be slidably disposed within working channel 108. A proximal handle (not shown) may be used to position and/or lock injection device 106 with respect to catheter 102. For example, catheter 102 may be positioned at a target location within a body lumen, for example, pylorus P, at which point injection device 106 may be distally extended with respect to catheter 102 so as to pierce a tissue of the body lumen, for example, pyloric sphincter PS. While not illustrated for the sake of simplicity in the drawings, it will be understood that an injection needle may define one or more channels extending therethrough, through which fluid may be distally delivered and/or proximally withdrawn, for example, via an operation of and/or application of pressure via a proximal handle.

While Figures of the present disclosure illustrate, by way of example, positioning of catheter 102 within stomach S, it will be understood that a catheter 102 may alternatively be positioned from within duodenum D, or from an alternative body lumen (not shown). Injection and/or deposition of bulking agents as described herein may be performed via endoscopic, subcutaneous, and/or percutaneous procedure.

As is shown in FIG. 1B, catheter 102 with injection device 106 may be used to inject a fluid volumizing agent 104a into a tissue, and in many cases, into a pyloric tissue. A fluid volumizing agent 104a may, in accordance with various principles of the present disclosure, comprise a silk-based bulking agent configured to volumize a tissue into which it is injected. For example, with respect to FIG. 1B, pyloric sphincter PS may be seen to be distended by volumizing agent 104a with respect to a natural positioning of the tissue as illustrated in FIG. 1A. The distending of the pyloric sphincter PS will thus be seen to change an effective internal diameter of the pyloric sphincter PS in a resting, open position from “D1” to “D2.” D2 may be less than D1, for example, less than 100% of a magnitude of D1.

In accordance with various principles of the present disclosure, volumizing agent 104a may polymerize (i.e., self polymerize, or polymerize in response to a curing step) to form an in situ tissue scaffold within a tissue, which may be configured to degrade at a similar and/or substantially identical rate as a growth rate of the tissue. Accordingly, volumizing agent 104a may be configured to degrade at a same rate at which tissue may grow into the space previously occupied by the volumizing agent 104a, thereby resulting in a natural and permanent volumization of the tissue. Without wishing to be bound by any particular theory, it is thus believed that tissue altered by methods of injecting volumizing agents as disclosed herein may present reduced risk of migration, inflammation, and loss of occlusive function over time over traditional methods of injected occlusive means.

In examples in which volumizing agent 104a is not used to reduce D2 to 0% of D1 (i.e., when volumizing agent 104a is used to partially occlude pyloric sphincter PS), one or more additional injections of volumizing agent may be used to further reduce the effective internal diameter of pyloric sphincter PS. FIG. 1C illustrates an additional injection of a bulking into the pyloric sphincter PS. In particular, when a sufficient amount of volumizing agent 104a has been injected into pyloric sphincter PS, injection device 106 may be proximally withdrawn from pyloric sphincter PS and repositioned for an additional injection of volumizing agent 104b. As can be seen with respect to FIG. 1C, volumizing agent 104b may volumize a tissue into which it is injected so as to decrease the effective internal diameter of pyloric sphincter PS from D2 to “D3.” D3 may be less than D1, for example, less than 100% of a magnitude of D1, and/or less than D2, for example, less than 100% of a magnitude of D2. Accordingly, multiple injections of a bulking agent may be used to customize and/or further occlude a body lumen beyond the effect of a single injection.

It is presently contemplated that multiple injections of a bulking agent may be performed over time according to a patient's needs. For example, volumizing agent 104a may be injected into pyloric sphincter PS and visualized, for example, via ultrasound imaging. In the event that volumizing agent 104a is determined to sufficiently occlude pyloric sphincter PS, a procedure may be ended. However, a patient's metabolic needs may change over time. An additional injection of volumizing agent 104b may thus be disposed in pyloric sphincter PS in a subsequent procedure. Embodiments are not limited in this context.

While FIGS. 1A-1C illustrate one or more injections of bulking agent about a pyloric sphincter, it is presently contemplated that alternatively positioned injection(s) may be preferred in some examples. Without wishing to be bound by any particular theory, one or more injection sites about a circumference of a body lumen may be selected based on tissue properties and/or geometry of the tissue defining the body lumen, which may increase an effectiveness and/or longevity of an occlusive injection.

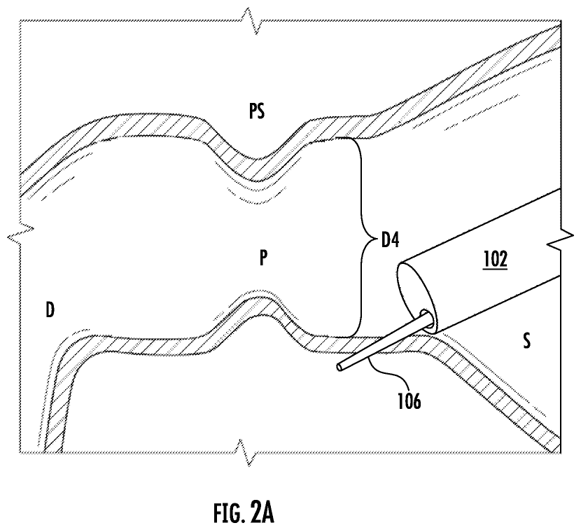

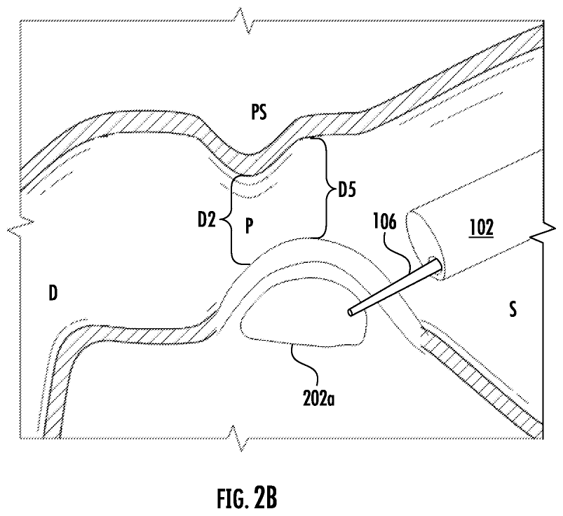

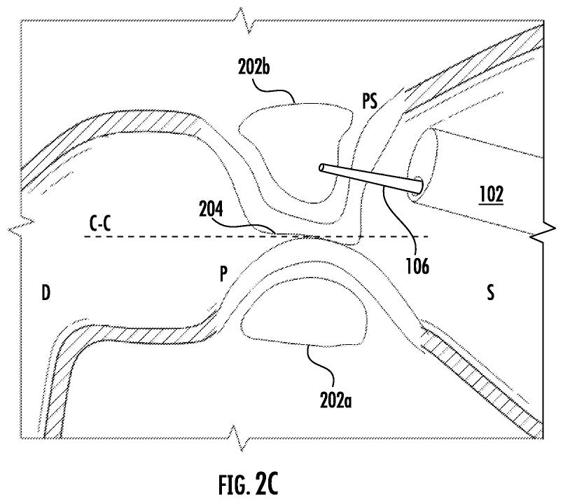

For example, FIG. 2A illustrates positioning of catheter 102 and injection device 106 for injection of a volumizing agent at a target location along a pyloric tissue adjacent to pyloric sphincter PS. According to examples of theories of the present disclosure, a tissue adjacent to pyloric sphincter PS may be understood to be less motile than pyloric sphincter PS itself (e.g., the adjacent tissue may not actively open and/or close with regular bodily function). Accordingly, an injection of a bulking agent into a tissue adjacent to pyloric sphincter PS may be subject to less force applied by surrounding tissue than a corresponding injection of a bulking agent into pyloric sphincter PS, and may, in various examples, be less subject to migration than a corresponding injection of a bulking agent into pyloric sphincter PS. Embodiments are not limited in this context.

FIG. 2B illustrates an injection of volumizing agent 202a at a target location as described with respect to FIG. 2A. As can be seen in FIG. 2B, volumizing agent 202a is deposited so as to extend under pyloric sphincter PS so as to affect an effective internal diameter of the same. However, it will be understood that, in alternative embodiments, volumizing agent 202a may be deposited entirely proximally to the pyloric sphincter PS so as to affect an effective internal diameter “D4” of pylorus P (illustrated in FIG. 2A) without affecting an internal effective diameter of pyloric sphincter PS. For example, D4 may be reduced to effective internal diameter “D5,” which may have a smaller magnitude than D4.

In FIG. 2C, an additional injection of volumizing agent 202b has been deposited in tissue of pylorus P adjacent to pyloric tissue PS. Volumizing agent 202b has been injected in FIG. 2C so as to substantially and/or completely occlude pylorus P, however, it will be understood that partial occlusions of pylorus P may alternatively be effected by volumizing agent 202b. As described above with respect to volumizing agent 202a, volumizing agent 202b may or may not extend to pyloric sphincter PS.

FIG. 2C additionally illustrates that one or more injections of volumizing agent may be deposited so as to create at least one substantially longitudinally extending (i.e., flat, planar, linearly extending, or the like) surface 204. One or more surfaces 204 may interface with each other or with other tissue defining a body lumen so as to more effectively seal the body lumen, for example, by increasing a surface area of contact along at least a longitudinal axis C-C extending through the body lumen.

Multiple injections of bulking agents may be used to create continuous and/or discontinuous bulking about a body lumen.

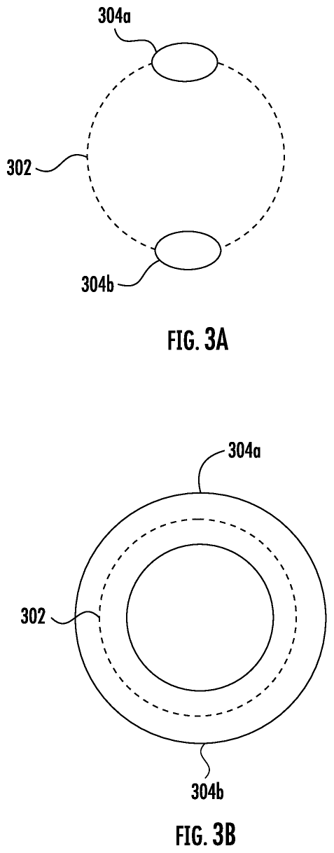

For example, as illustrated in FIG. 3A, circumference 302 is an example of a cross sectional circumference of pyloric sphincter PS as illustrated in FIGS. 1A-1C and/or FIGS. 2A-2C, taken along plane B-B as illustrated in FIG. 1C. As illustrated in FIG. 3A, injections of volumizing agent 304a and volumizing agent 304b may be discontinuously disposed along circumference 302 of pylorus sphincter PS. While FIG. 3A illustrates two injections arranged diametrically along circumference 302, it will be understood that additional injections of bulking agent may be additionally and/or alternatively disposed along circumference 302. For example, three or more injections may be equally or unequally spaced along circumference 302. In another example, injection of volumizing agent 304b may be offset along circumference 302. In yet another example, injections of volumizing agent 304a, 304b may be injected at different depths with respect to each other. In yet another example, injections of volumizing agents 304a, 304b may comprise different volumes from each other. In yet another example, injections of volumizing agents 304a, 304b may comprise different shapes from each other.

Injections of volumizing agents 304a, 304b as illustrated in FIGS. 3A-3B may be understood to refer to examples of either volumizing agents 104a, 104b or bulking agents 404a, 404b as described below.

Accordingly, various injections of bulking agent may be used to customize a full or partial occlusion of a body lumen based on unique patient anatomy and procedural objectives (i.e., percentage of occlusion of the pyloric sphincter, which may correspond with a percentage of difference between D1 and D2 and/or D3.

In some embodiments, multiple injections of bulking and/or volumizing agents may be used to create one or more continuous implants about circumference 302. For example, in FIG. 3B, volumizing agent 304a and volumizing agent 304b have been injected about circumference 302 so as to continuously extend about circumference 302 and form a single effective occlusive implant. While FIG. 3B illustrates a uniform deposition of bulking agent about circumference 302 for the sake of simplicity, it will be recognized that bulking agent may be disposed about circumference 302 asymmetrically (i.e., with different depths, volumes, and/or shapes).

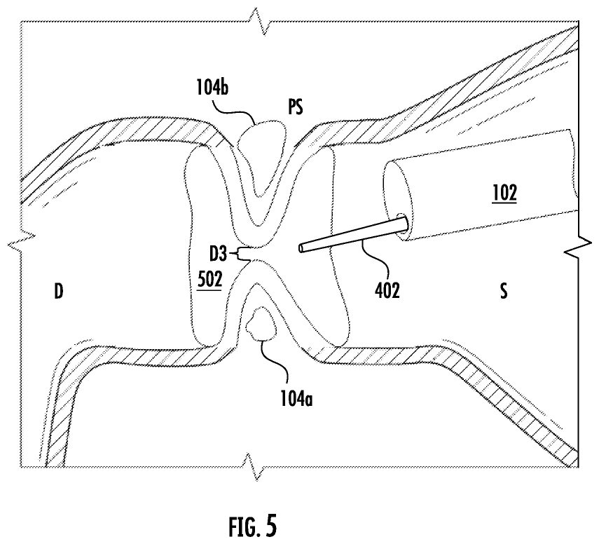

One will understand that arrangements of bulking agents described herein may additionally and/or alternatively apply to injections described with respect to FIGS. 1A-1C, FIGS. 2A-2C, FIG. 4, and/or FIG. 5, for example, of volumizing agent 104a, 104b, 202a, and/or 202b, and/or bulking agent 404a, 404b, as discussed below.

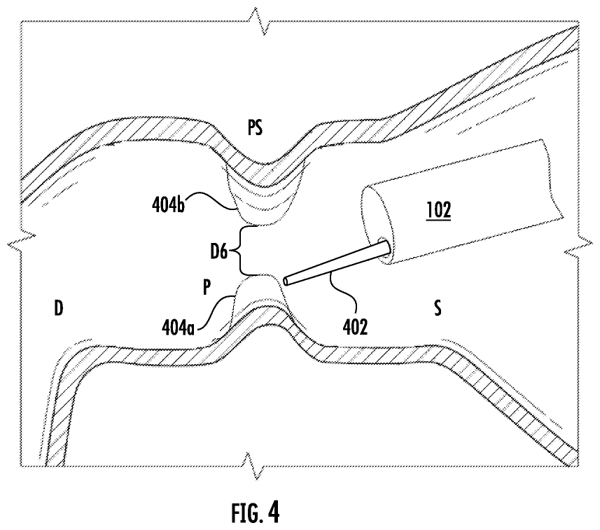

In accordance with various aspects of the present disclosure, a bulking agent may be disposed on or along a surface of a tissue defining a body lumen such as by injecting the agent on or along the surface. For example, in FIG. 4, catheter 102 may be used to position injection device 402, which may comprise one or more similarities to injection device 106, to dispose one or more deposits of bulking agent 404a, 404b on or over an inner surface of a tissue defining a body lumen, for example, on pyloric sphincter PS, to create at least one in situ occlusive implant. In some embodiments, bulking agent 404a, 404b may be an adhesive fluid which may self-polymerize or be otherwise cured to create a solid bolus on or along a surface to which it has been applied. In some cases, bulking agent 404a, 404b may comprise a multipart adhesive with at least first and second components which, when exposed to each other, react to solidify. In some embodiments, bulking agent 404a, 404b may comprise a fibrin glue with a first part comprising fibrinogen and a second part comprising thrombin.

It is presently contemplated that one or more depositions of bulking agent 404a, 404b on or along a tissue surface may allow for comparatively easier removal thereof as opposed to, for example, injection into the tissue surface, which may enable greater accommodation of a patient's needs over time. For example, as discussed above, a patient's needs may develop over time, for example, with respect to weight loss and/or metabolic activity. In some embodiments, injected volumizing agents may accommodate substantially permanent effects to an effective inner diameter of a body lumen, as removal of the injected volumizing agent and/or removal of ingrown tissue may be difficult or impossible. However, bulking agent 404a, 404b deposited on or along a tissue surface as illustrated in FIG. 4 will be recognized to be more easily resectable, for example, via a cutting element deployed via catheter 102 (not shown). Accordingly, methods of treatment involving bulking agent 404a, 404b may comprise, after deposition and/or gelation of bulking agent 404a, 404b, positioning catheter 102 near at least one resultant injected implant, and/or using a cutting element deposed on a distal tip of catheter 102 to resect at least part of the at least one resultant injected implant. Accordingly, an effective inner diameter of a body lumen may be increased, for example, from D6 to D1, or any diameter in between.

In some embodiments, injection device 402 may comprise a multi-channel needle, such as a dual-channel needle. If injection device 402 comprises multiple channels, each channel may be configured to facilitate equal and/or otherwise coordinated flow of fluid therethrough. For example, injection device 402 may be configured to deliver equal parts of fibrinogen and thrombin, or a steady ratio of components, such as a 40%-60% ratio, or a 30%-70% ratio, or another ratio, of a first part to second part.

One or more deposits of bulking agent 404a, 404b may reduce an effective inner diameter of a body lumen, thereby partially or completely occluding the body lumen. For example, as illustrated in FIG. 4, deposits of bulking agent 404a, 404b may reduce an effective inner diameter of pyloric sphincter PS from D1, as illustrated in FIG. 1A, to effective inner diameter “D6.” D6 may be less than D1, for example, less than 100% of a magnitude of D1.

Multiple deposits of bulking agent 404a, 404b may be deposited on or along a tissue surface in a single procedure or in multiple procedures, as discussed with respect to volumizing agents 104a, 104b above.

According to one or more examples of the present disclosure, at least one deposit of bulking agent on or along a tissue surface may be used to substantially and/or entirely occlude a body lumen. In some examples of the present disclosure, at least one deposit of bulking agent may be deposited selectively on or along a tissue surface according to tissue properties and/or geometry of the tissue defining the body lumen, which may increase an effectiveness and/or longevity of an occlusive injection.

It will be appreciated that various principles, devices, systems, and methods described herein with respect to volumizing agents and bulking agents may be used in conjunction with each other. For example, as illustrated in FIG. 5, injected volumizing agents and deposited bulking agents may be used together to customize an occlusion of a body lumen. Volumizing agent 104a, 104b may be injected into a pyloric tissue, for example, pyloric sphincter PS, to partially or fully occlude pylorus P (i.e., reducing an effective inner diameter thereof from D1 to D3). In another step, implant 502 may be created in situ by disposing a bulking agent on or along a surface of the pyloric tissue. Each of volumizing agent 104a, 104b, and/or the bulking agent used to form in situ implant 502 may be the same or a different fluid bulking agent. In various examples, volumizing agent 104a, 104b may comprise a silk-based bulking agent and implant 502 may be formed from a fibrin glue.

In some embodiments, volumizing agent 104a, 104b may be injected prior to formation of implant 502, which may in some examples enable a pyloric tissue to be positioned so as to better facilitate adherence of a bulking agent on or along a surface thereof in order to form implant 502. In various embodiments, implant 502 may be formed prior to injection of volumizing agent 104a, 104b, which may in some examples contribute to a stabilization or propping of the pyloric tissue so as to enable more precise placement of volumizing agent 104a, 104b. In some embodiments in which implant 502 is formed prior to injection of volumizing agent 104a, 104b, implant 502 may be formed with one or more pores extending to a tissue surface in order to facilitate easier injection of volumizing agent 104a, 104b (not shown).

While FIG. 5 is illustrated with respect to arrangements of volumizing agent 104a, 104b as described with respect to FIGS. 1A-1C, one will understand that arrangements of volumizing agent 202a, 202b as described with respect to FIGS. 2A-2C may alternatively or additionally used. Volumizing agent and/or bulking agent may be injected and/or disposed anywhere along a tissue defining a body lumen so as to effectively alter a diameter of the body lumen.

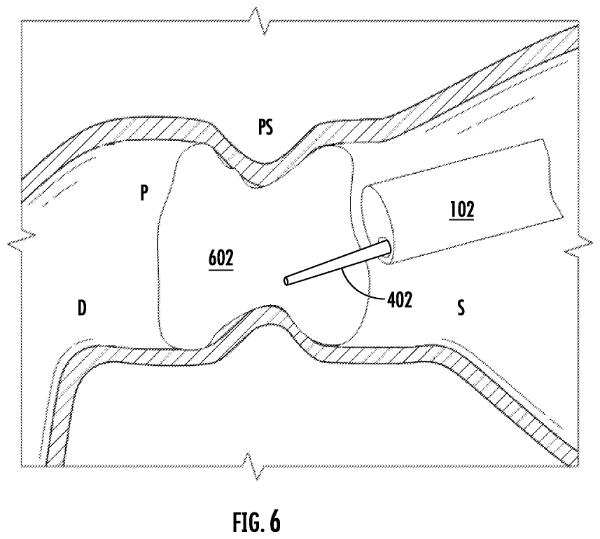

It will be appreciated that various principles, devices, systems, and methods of the present disclosure with respect to depositing of bulking agents on a surface of a body tissue to form an situ implant may be used in a variety of applications beyond those described with respect to use of a silk-based bulking agent. For example, FIG. 6 illustrates deposition of bulking agent across a lumen defined by pyloric sphincter PS, and additionally at least somewhat extending along pyloric tissue adjacent to the pyloric sphincter PS. The bulking agent thus forms in situ implant 602, which may fully occlude pylorus P. The surrounding of pyloric sphincter PS by implant 602 may increase a resistance to migration of and/or unintended detachment of implant 602 from surrounding tissue by anchoring of implant 602 to at least one side of pyloric sphincter PS and/or to tissue adjacent to pyloric sphincter PS, for example, as opposed to depositions of bulking agent 404a, 404b arranged as described with respect to FIG. 4, which cap pyloric sphincter PS but which to not extend along its full side or along adjacent tissue.

Adhesive properties of deposited bulking agent may be understood to improve a resistance of implant 602 to migration such as by anchoring implant 602 with respect to the site at which the agent is deposited, for example, as opposed to a stent or other occlusive device delivered and positioned within a body lumen without binding to surrounding tissue immediately upon implantation (not shown). Furthermore, the in situ formation of implant 602, such as through use of one or more injectable agents, may accommodate and/or conform to a natural shape of surrounding tissue as the bulking agent solidifies, thereby accommodating and/or conforming to a shape of the surrounding tissue more exactly than a preformed implant. Accordingly, implant 602 may enable better customization of treatment to a patient's unique anatomical variations over various traditional occlusive implants.

Benefits of combinations of injected and deposited volumizing/bulking agents may extend beyond placement and/or forming of the respective resultant implants. In various examples according to the present disclosure, combinations of injected and deposited volumizing/bulking agents may enable customization of care for a patient which accounts for potential changes in patient needs over time. For example, volumizing agent 104a, 104b may be injected to an extent that a permanent reduction in body lumen diameter may be predicted to be necessary or acceptable, and implant 502 may be formed to affect the body lumen diameter according to needs which may be temporary. A first agent and a second agent may be deposited along the surface of a tissue, the agents being configured to react with each other to polymerize and form an intraluminal occlusion device on the tissue surface and anchored to the tissue. Methods of treatment disclosed herein may thus provide improvements in customization and use over traditional occlusive methods.

The same or a different injection device 106, 402 may be used to inject and/or deposit volumizing agent 104a, 104b and implant 502, 602. For example, a multi-channel injection device 402 may be used which includes channels for each component of implants 502, 602 and for delivery of volumizing agent 104a, 104b. Alternatively, separate needles may be used in alternative working channels 108 of catheter 102, or from a single working channel 108 (i.e., injection device 106 may be used to inject volumizing agent 104a, 104b, then injection device 106 may be removed from working channel 108 and replaced with injection device 402 to form implant 502, 602).

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit, and scope of the disclosure. All such similar substitutes and modifications apparent to those skilled in the art are deemed to be within the spirit, scope and concept of the disclosure as defined by the appended claims.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the term “comprises/comprising” does not exclude the presence of other elements or steps. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A pyloric occlusion system comprising a silk-based tissue volumizing agent injected into a pyloric tissue, wherein the silk-based tissue volumizing agent is configured to degrade at approximately a same rate as a growth rate of the pyloric tissue.

2. The pyloric occlusion system of claim 1, wherein the silk-based tissue volumizing agent is configured to partially occlude a pylorus.

3. The pyloric occlusion system of claim 1, further comprising a first bulking agent component and a second bulking agent component configured to polymerize when placed in contact in-situ with the first bulking agent component on a surface of the pyloric tissue, wherein the first bulking agent component is fibrinogen, the second bulking agent component is thrombin, or both.

4. The pyloric occlusion system of claim 3, wherein the first bulking agent component and the second bulking agent component are configured to polymerize to form an in-situ implant.

5. The pyloric occlusion system of claim 3, wherein the in-situ implant is configured to completely occlude the pylorus.

6. The pyloric occlusion system of claim 3, wherein the in-situ implant is configured to anchor to at least one side of the pyloric sphincter.

7. The pyloric occlusion system of claim 1, wherein the silk-based tissue volumizing agent is imageable via ultrasound.

8. A system for occluding a pylorus, comprising:
an injection device configured to inject medium into or adjacent a pylorus; and
an injectable pyloric occlusion medium comprising a silk-based bulking agent.

9. The system of claim 8, wherein the silk-based bulking agent has a degradation rate approximately the same as a growth rate of an apposed tissue.

10. The system of claim 8, further comprising a:
a first agent; and
a second agent;
wherein the first agent and the second agent are configured to react with each other to polymerize and form an intraluminal occlusion device on the surface of the pylorus, the occlusion device anchored to at least one side of the pyloric sphincter.

11. A system for occluding a pylorus, the system comprising:
a volumizing agent;
a first bulking agent component;
a second bulking agent component configured to polymerize when placed in contact with the first bulking agent component; and
at least one injection device, comprising at least one channel extending therethrough, the at least one injection device configured to inject the volumizing agent into a tissue and to deposit the first bulking agent component and the second bulking agent component onto the tissue surface.

12. The system of claim 11, wherein the tissue is a pylorus.

13. The system of claim 11, wherein the first bulking agent component is thrombin and the second bulking agent component is fibrinogen.

14. The system of claim 11, wherein the volumizing agent is configured to degrade at approximately a same rate as a growth rate of the tissue.

15. The system of claim 11, wherein the volumizing agent is configured to, when injected into the tissue, augment the tissue.

16. The system of claim 11, wherein the tissue volumizing agent is a silk-based volumizing agent.

17. The system of claim 16, wherein the tissue volumizing agent is configured to, when injected into the tissue, effect a partial occlusion of the lumen.

18. The system of claim 16, wherein the first and second bulking agent components are configured to polymerize to form an in-situ implant.

19. The system of claim 18, wherein the in-situ implant is configured to fully occlude the lumen.

20. The system of claim 18, wherein the in-situ implant is removable.
